(19) United States
(12) Patent Application Publication
(10) Pub. No.: US 2022/0020478 A1
(43) Pub. Date: Jan. 20, 2022

(54) SYSTEM AND METHOD FOR IDENTIFYING LOW CLINICAL VALUE TELEMETRY CASES

(71) Applicant: KONINKLIJKE PHILIPS N.V., EINDHOVEN (NL)

(72) Inventors: David Paul Noren, Cambridge, MA (US); Lasith Adhikari, Revere, MA (US); Gregory Boverman, Reading, MA (US); Rinku Skaria, Cambridge, MA (US)

(21) Appl. No.: 17/235,994

(22) Filed: Apr. 21, 2021

Related U.S. Application Data
(60) Provisional application No. 63/051,919, filed on Jul. 15, 2020.

(51) Int. Cl. G16H 40/20; G16H 50/20; G16H 70/20; G16H 10/60; G06N 5/00

(52) U.S. Cl. CPC G16H 40/20; G16H 50/20; G06N 5/003; G16H 10/60; G16H 70/20

(57) ABSTRACT

A method for generating a telemetry indication score for a patient using a telemetry analysis system, comprising: (i) receiving, by the telemetry analysis system, medical information about the patient comprising one or more patient demographics, one or more physiological measurements, and/or a patient diagnosis; (ii) analyzing the received medical information using a decision support tool, wherein the decision support tool utilizes telemetry guidelines; (iii) determining, by a trained machine learning algorithm using the results of the decision support tool, a telemetry indication score for the patient comprising a probability of whether the patient is likely to meet the telemetry guidelines; and (iv) providing, via a user interface, a telemetry indication report for the patient, wherein the telemetry indication report comprises the telemetry indication score and further wherein the telemetry indication report comprises evidence supporting the telemetry indication score.

### FIELD OF THE DISCLOSURE

The present disclosure is directed generally to methods and systems for predicting whether telemetry is indicated or contraindicated for a patient.

### BACKGROUND

Centralized monitoring of non-critically ill cardiac patients from a remote facility—Central Monitoring Unit (CMU)—is no longer a new concept in healthcare. More than 60% of U.S. hospitals have been using CMUs for at least a decade. Specially trained telemetry monitoring technicians in CMUs are responsible for both interpreting cardiac rhythms and responding to alarms with minimal delay. Monitoring 24 to 60 patients at a time with 350 to 700 alarms per patient per day for 8 to 12 hours continuously is a challenging and stressful job, and life-threatening events might be missed due to fatigue or stress. This alarm burden directly impacts technicians, leading to annoyance, anxiety, low job satisfaction, and burn-out.

In addition to alarm fatigue, over-prescription of telemetry services can unnecessarily utilize resources. Indeed, over-prescription of telemetry services has become a severe problem in many hospitals. Patients are commonly prescribed telemetry monitoring as a means of additional oversight of patient vital signs rather than to address a specific cardiac rhythm concern. Telemetry overuse can be conceptually separated into two types. The first type is the initiation of telemetry for patients that do not present with evidence of cardiac risk requiring monitoring of cardiac rhythms. The second type is the continued use of telemetry for patients that initially presented with valid indications for telemetry but no longer warrant monitoring. As a result of this misuse, hospital telemetry services become overburdened, experiencing shortages in telemetry monitoring devices. In response to this, the American Heart Association (AHA) has published a set of evidence-based guidelines that specify a consensus of accepted telemetry use cases and provide guidance in how to determine if a patient meets these conditions. Unfortunately, these guidelines are extremely complex and contextual. In addition, the information needed for the decision criteria is often incomplete, making interpretation challenging and identification of cases that do not conform to guidelines very difficult. This presents a significant barrier, if not impossibility, for hospitals to rely on understanding, adoption, and continuous execution among care providers as a means of enforcing the valid initiation and continuation of telemetry. Constant re-evaluation is required to determine when a patient can be taken off telemetry, which is very time consuming. Additionally, the evaluation of telemetry usage requires communication among multiple roles and cannot be accomplished by a clinician alone, e.g., a doctor needs to communicate with a nurse and telemetry technician to determine if the patient has been asymptomatic and alarm free, respectively.

### SUMMARY OF THE DISCLOSURE

There is a continued need for methods and systems that evaluate the need for telemetry monitoring for patents in order to reduce or prevent alarm fatigue, to maximize hospital resources, and to lead to improved patient outcomes.

The present disclosure is directed at inventive methods and systems for generating a telemetry indication score for a patient using a telemetry analysis system. Various embodiments and implementations herein are directed to a system or method that receives medical information about a patient including at least one or more patient demographics, one or more physiological measurements, and a patient diagnosis. The telemetry analysis system analyzes the medical information using a decision support tool that comprises a set of telemetry guidelines. A trained machine learning algorithm of the telemetry analysis system uses the results of the decision support tool analysis to determine a telemetry indication score for the patient which comprises a probability of whether the patient is likely to meet the telemetry guidelines. The telemetry analysis system provides a telemetry indication report for the patient via a user interface. The telemetry indication report comprises the telemetry indication score, as well as evidence supporting the telemetry indication score.

Generally, in one aspect, a method for generating a telemetry indication score for a patient using a telemetry analysis system is provided. The method includes: (i) receiving, by the telemetry analysis system, medical information about the patient comprising one or more patient demographics, one or more physiological measurements, and/or a patient diagnosis; (ii) analyzing the received medical information using a decision support tool, wherein the decision support tool utilizes telemetry guidelines; (iii) determining, by a trained machine learning algorithm using the results of the decision support tool, a telemetry indication score for the patient comprising a probability of whether the patient is likely to meet the telemetry guidelines; and (iv) providing, via a user interface, a telemetry indication report for the patient, wherein the telemetry indication report comprises the telemetry indication score and further wherein the telemetry indication report comprises evidence supporting the telemetry indication score.

According to an embodiment, the method further includes the step of programming the decision support tool, comprising: (i) receiving one or more telemetry guidelines; (ii) generating one or more decision trees utilizing the received one or more telemetry guidelines; (iii) optionally adding or modifying, by a healthcare professional, one or more elements of one or more decision trees.

According to an embodiment, the method further includes the step of training the machine learning algorithm, comprising: (i) receiving a dataset of historical patient data, the dataset comprising for each of a plurality of patient medical information about the patient and telemetry monitoring data for the patient; (ii) extracting, using the decision support tool, a plurality of features from the patient medical information and telemetry monitoring data; and (iii) training the machine learning algorithm using the extracted features.

According to an embodiment, the decision support tool comprises a plurality of decision trees each comprising a plurality of decision points derived from telemetry guidelines. According to an embodiment, the decision support tool is configured to determine for the patient, based on the decision tree, one or more elements within the decision tree indicating telemetry and one or more elements within the decision tree contraindicating telemetry.

According to an embodiment, the decision support tool is configured to determine for the patient a percentage of elements within the decision tree indicating telemetry.

According to an embodiment, the decision support tool is configured to determine which of the plurality of decision trees to utilize for the analysis.

According to an embodiment, the one or more physiological measurements comprises one or more vital signs, one or more clinical test results, and/or one or more cognitive assessments.

According to an embodiment, the patient diagnosis comprises one or more medical diagnoses, one or more comorbidities, and/or one or more historical medical records.

According to an embodiment, the trained machine learning algorithm is further configured to determine a confidence score for the telemetry indication score, and wherein the telemetry indication report for the patient further comprises the confidence score.

According to an embodiment, the telemetry indication score is a number between 1 and 100.

According to another aspect is a telemetry analysis system configured to generate a telemetry indication score for a patient. The system includes: a database comprising medical information about the patient, comprising one or more patient demographics, one or more physiological measurements, and/or a patient diagnosis; a decision support tool, wherein the decision support tool utilizes telemetry guidelines; a classifier trained to generate a telemetry indication score for the patient comprising a probability of whether the patient is likely to meet the telemetry guidelines; a processor configured to: (i) receive the medical information from the database; (ii) direct the decision support tool to analyze the received medical information; (iii) direct the classifier to determine the telemetry indication score for the patient; and (iv) generate a telemetry indication report for the patient; and a user interface configured to provide the generated telemetry indication report for the patient, wherein the telemetry indication report comprises the telemetry indication score and further wherein the telemetry indication report comprises evidence supporting the telemetry indication score.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for generating a telemetry indication score for a patient using a telemetry analysis system, in accordance with an embodiment.

FIG. 2 is a flowchart of a method for generating a trained classifier, in accordance with an embodiment.

FIG. 3 is a flowchart of a decision tree of a decision support tool configuration, in accordance with an embodiment.

FIG. 4 is a table of information accompanying the decision tree of FIG. 3, in accordance with an embodiment.

FIG. 5 is a flowchart of a decision tree of a decision support tool configuration, in accordance with an embodiment.

FIG. 6 is a table of information accompanying the decision tree of FIG. 5, in accordance with an embodiment.

FIG. 7 is a flowchart of a decision tree of a decision support tool configuration, in accordance with an embodiment.

FIG. 8A is a table of information accompanying the decision tree of FIG. 7, in accordance with an embodiment.

FIG. 8B is a table of information accompanying the decision tree of FIG. 7, in accordance with an embodiment.

FIG. 9 is a flowchart of a decision tree of a decision support tool configuration, in accordance with an embodiment.

FIG. 10A is a table of information accompanying the decision tree of FIG. 9, in accordance with an embodiment.

FIG. 10B is a table of information accompanying the decision tree of FIG. 9, in accordance with an embodiment.

FIG. 11 is a schematic representation of a telemetry analysis system, in accordance with an embodiment.

### DETAILED DESCRIPTION OF EMBODIMENTS

The present disclosure describes various embodiments of a system and method for generating a telemetry indication score for a patient using a telemetry analysis system. Applicant has recognized and appreciated that it would be beneficial to provide a method and system that can more accurately and more quickly provide an analysis regarding whether telemetry is indicated or contraindicated for a patient, in order to reduce or prevent alarm fatigue, to maximize hospital resources, and to lead to improved patient outcomes. The telemetry analysis system receives medical information about a patient including patient demographics, physiological measurements, medical history, and/or patient diagnosis. The telemetry analysis system analyzes the medical information using a decision support tool that comprises a set of telemetry guidelines. A trained machine learning algorithm of the telemetry analysis system uses the results of the decision support tool analysis to determine a telemetry indication score for the patient which comprises a probability of whether the patient is likely to meet the telemetry guidelines. The telemetry analysis system provides a telemetry indication report for the patient via a user interface. The telemetry indication report comprises the telemetry indication score, as well as evidence supporting the telemetry indication score.

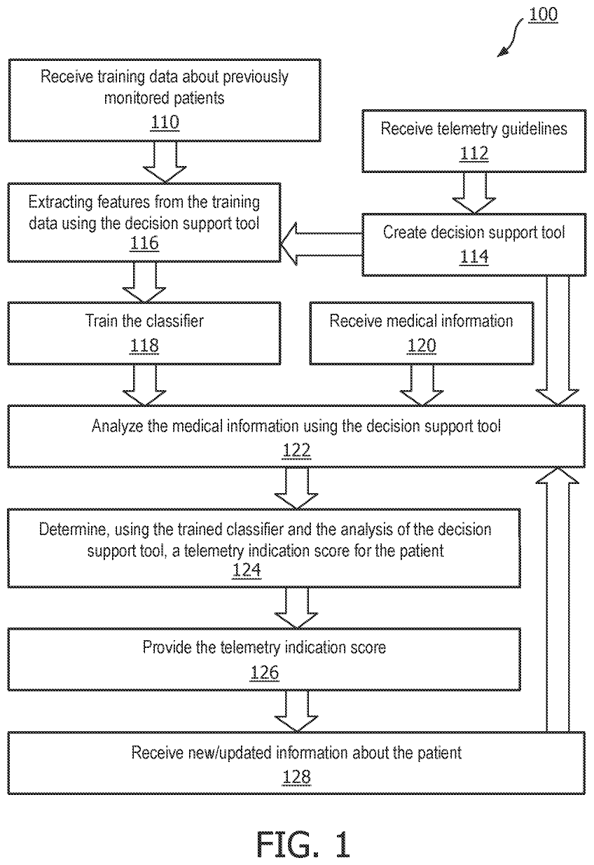

Referring to FIG. 1, in one embodiment, is a flowchart of a method 100 for generating a telemetry indication score for a patient using a telemetry analysis system. The telemetry analysis system may be any telemetry analysis system described or otherwise envisioned herein.

As discussed in greater detail herein, the telemetry analysis system comprises a decision support tool that comprises telemetry guidelines serving as a mechanism to determine whether a patient should undergo telemetry monitoring. The guidelines utilized to program or generate the decision support tool can be the American Heart Association guidelines as they are written now or as updated in the future, guidelines from another similar organization, guidelines created by any healthcare professional or healthcare organization, or any combination thereof. Additionally, the guidelines can be personalized or customized for a particular use or location.

Also as discussed in greater detail herein, the telemetry analysis system comprises a machine learning algorithm that has been trained to use the results of the decision support tool to determine a probability of whether the patient is likely to meet the telemetry guidelines. The machine learning algorithm of the telemetry analysis system can be trained using a dataset of historical patient data, the dataset comprising for each of a plurality of patient medical information about the patient and telemetry monitoring data for the patient. Features are extracted from the historical patient medical information and telemetry monitoring data using the decision support tool, and the machine learning algorithm can be trained using the extracted features.

Accordingly, at step 110 of the method, the telemetry analysis system receives input data comprising medical information about a plurality of monitored patients. The medical information includes patient demographics, physiological measurements, historical health records, and/or a patient diagnosis, as well as telemetry monitoring data for each patient. The input data may be stored in and/or received from one or more databases. The database may be a local and/or remote database. For example, the monitoring center or telemetry analysis system may comprise a database of input data such as a medical health record database among many other types of databases.

In order to train the machine learning algorithm of the telemetry analysis system, the input can comprise a wide variety of input types in addition to the telemetry monitoring data for each patient. As an example, the input data can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or telemetry alarm related data such as arrhythmia-related alarms (ventricular tachycardia, ventricular bradycardia, atrial fibrillation, asystole, and more), physiologic alarms such as heart rate, respiratory rate, apnea, SpO₂, invasive arterial pressure, noninvasive blood pressure, and more. Many other types of input data are possible.

According to an embodiment, the telemetry analysis system may comprise a data pre-processor or similar component or algorithm configured to process the received input data. For example, the data pre-processor analyzes the input data to remove noise, bias, errors, and other potential issues. The data pre-processor may also analyze the input data to remove low quality portions of ECG waveform data. The data pre-processor may extract ECG event codes to indicate whether specific arrythmias have been detected. Telemetry alarm data may also be extracted by the data pre-processor. Many other forms of data pre-processing or data point identification and/or extraction are possible.

At step 112 of the method, which can occur before or after step 110 of the method, telemetry guidelines are received in order to create the decision support tool of the telemetry analysis system. The guidelines can be any guidelines, instructions, preferences, or other type of information or guidance that is intended to facilitate, direct, and/or support telemetry monitoring decisions. For example, the guidelines can be the American Heart Association guidelines as they are written now or as updated in the future, guidelines from another similar organization, guidelines created by any healthcare professional or healthcare organization, or any combination thereof. The guidelines may be stored in and/or received from one or more databases. The database may be a local and/or remote database. For example, the monitoring center or telemetry analysis system may comprise a database of guidelines.

At step 114 of the method, a decision support tool of the telemetry analysis system is created using the received telemetry guidelines. The decision support tool may be of any configuration, design, or format that enables comparison of telemetry input data to elements of the received telemetry guidelines. For example, according to just one embodiment, the decision support tool may be or comprise one or multiple decision trees in which elements of the received telemetry guidelines are formatted into nodes of the decision trees. Thus, medical information received about a patient can be processed through the decision trees determining which elements are met or not met, how well an element is met, and/or other data about the elements in order to create an output of the decision support tool. Other examples of decision support tools are checklists, among many others. Non-limiting examples of a decision support tool are provided elsewhere herein. The decision support tool may of course be customized and updated as clinical practice evolves.

According to an embodiment, the configuration, design, or format of the decision support tool may be modified by a healthcare professional, clinician, technician, IT professional, or others in order to adjust the decision support tool. For example, a healthcare professional may add an element to a decision tree, or the healthcare professional may modify or remove an element from a decision tree. This manual modification may be based on updated guidance, personal preference, experience, new data, new machinery, and/or any other reason for modifying the decision flow for analyzing telemetry monitoring.

At step 116 of the method, the system extracts features from the received training data using the generated decision support tool. This can be accomplished by a variety of embodiments for feature identification, extraction, and/or processing, including any method for extracting features from a dataset. According to an embodiment, the telemetry analysis system may comprise a data transformer or similar component or algorithm configured to process the received dataset. For example, the data transformer can process the input data through the guideline-based decision support tool and can determine whether a condition specified by each decision element have been met. The data transformer utilizes this information to derive the features used to train the machine learning model. The information can include things such as the specific elements present, the percentage of each decision path that has positive induction, the number of paths that have a single indication, the number of trees that are implicated by the data, the maximum level in any decision tree that has a positive indicator, and much more.

The outcome of a feature processing step or module of the telemetry analysis system is a set of features related to telemetry monitoring need for each of a plurality of previously monitored patients, which thus comprises a training data set that can be utilized to train the classifier.

At step 118 of the method, the system trains the machine learning algorithm, which will be the classifier utilized to analyze medical information from a patient as described or otherwise envisioned herein. The machine learning algorithm is trained using the extracted features according to known methods for training a machine learning algorithm. According to an embodiment, a machine learning classifier such as random forest, XGBoost, and/or any other classifier can be used to learn the patient clinical phenotypes and monitoring needs of the patient. Many other classifiers are possible.

Following step 118, the telemetry analysis system comprises a trained classifier that can be utilized to classify telemetry analysis and provide a telemetry indication score for the patient comprising a probability of whether the patient is likely to meet the telemetry guidelines. The trained classifier can be static such that it is trained once and is utilized for classifying. According to another embodiment, the trained classifier can be more dynamic such that it is updated or re-trained using subsequently available training data. The updating or re-training can be constant or can be periodic.

With a decision support tool and trained classifier, the telemetry analysis system is ready to classify patient data and provide telemetry indication scores. Accordingly, at step 120 of the method, the telemetry analysis system medical information for a patient to be analyzed by the system. The medical information about the patient includes patient demographics, physiological measurements, historical health records, and/or a patient diagnosis. Preferably, the received information comprises data relevant to the decision support tool utilized by the system, in order to maximize the accuracy of the decision support tool and classifier. This medical information may be stored in and/or received from one or more databases. The database may be a local and/or remote database. For example, the monitoring center or telemetry analysis system may comprise a database of medical information such as a medical health record database among many other types of databases.

According to an embodiment, the received medical information may be processed by the system such that it can be utilized by the decision support tool. For example, the system may identify, extract, and process one or more features or data points for each patient for use by the decision support tool. Features may be utilized by the decision support tool immediately or may be stored for downstream or later use by the system.

At step 122 of the method, the telemetry analysis system analyzes the received medical information using a decision support tool. For example, the data transformer can process the medical information through the guideline-based decision support tool and can determine whether a condition specified by each decision element have been met. The information generated by the data transformer can include things such as the specific elements present, the percentage of each decision path that has positive induction, the number of paths that have a single indication, the number of trees that are implicated by the data, the maximum level in any decision tree that has a positive indicator, and much more. The outcome of the data transformer at step 122 of the method is therefore a dataset comprising information about the patient's condition relative to the telemetry monitoring guidelines of the decision support tool.

At step 124 of the method, the telemetry analysis system processes the data derived by the decision support tool in step 122 using the trained classifier. The trained classifier is configured, as described herein, to generate a telemetry indication score for the patient comprising a probability of whether the patient is likely to meet the telemetry guidelines. This telemetry indication score and probability are based on the extensive historical dataset utilized to train the classifier. According to an embodiment, the trained classifier can be further configured to determine a confidence level, score, or indication for the telemetry indication score.

According to an embodiment, the telemetry indication score is a score between 1-100 indicating whether a patient is likely to meet the guideline criteria for telemetry usage based on the available evidence. Alternatively, a threshold can be established under which the clinical case is considered low value and a binary recommendation, i.e. “high value” or “low value”, can be provided. Many other variations of a telemetry indication score are possible.

The telemetry indication score generated by the trained classifier of the telemetry analysis system is packaged or otherwise added to a telemetry indication report for the patient. The telemetry indication report may be a standard format into which output data from the classifier is populated. The telemetry indication report may also be dynamic and formatted or configured based on the output of the classifier.

At step 126 of the method, the generated telemetry indication report for the patient is provided via a user interface of the telemetry analysis system. The generated telemetry indication report comprises the telemetry indication score generated by the trained classifier of the telemetry analysis system. According to an embodiment, the generated telemetry indication report comprises one or more pieces of evidence supporting or informing the telemetry indication score. For example, the evidence may comprise information such as the specific elements of the guidelines present, the percentage of each decision path that has positive induction, the number of paths that have a single indication, the number of decision trees that are implicated by the data, the maximum level in any decision tree that has a positive indicator, and much more. According to an embodiment, the telemetry indication report for the patient further comprises the confidence score.

The generated telemetry indication report for the patient can be provided via any mechanism, and via any user interface. The user interface may include one or more devices for enabling communication with a user. The user interface can be any device or system that allows information to be conveyed and/or received, and may include a display, a mouse, and/or a keyboard for receiving user commands. The user interface may be located with one or more other components of the system, or may located remote from the system and in communication via a wired and/or wireless communications network.

According to an embodiment, the generated telemetry indication report for the patient can then be used by a care provider to determine which patient cases should be reviewed for a telemetry decision, such as whether to initiate telemetry, and whether to continue or discontinue telemetry.

At optional step 128 of the method, the telemetry analysis system receives updated medical information about the patient. This may include any of the types of information described or otherwise envisioned herein. For example, the system may receive updated vital information, diagnosis or prognosis information, previous telemetry data, and/or any other type of information relevant to telemetry monitoring decisions. Thus, the system may perform ongoing analysis of the patient to determine the indication or contraindication of telemetry monitoring.

With the received updated medical information about the patient, the system may return to step 122 of the method to analyze the updated medical information using the decision support tool. The method can then progress as described, leading to the generation of a new or updated telemetry indication report for the patient. This updated telemetry indication report may comprise a comparison to a previous report, including a comparison of the updated and one or more previous telemetry indication scores. The updated telemetry indication report may also comprise information or evidence indicating or supporting a change between the updated and one or more previous telemetry indication scores.

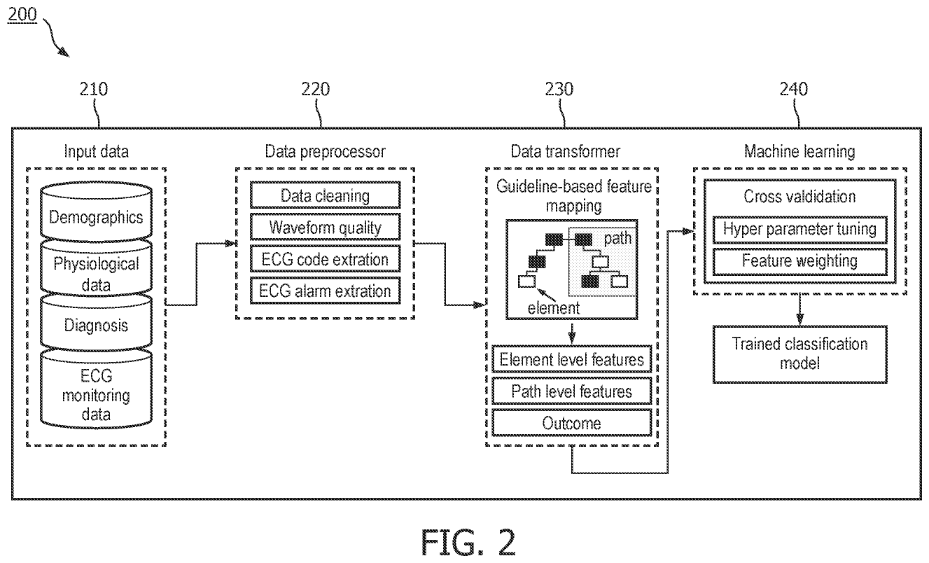

Referring to FIG. 2, in one embodiment, is a schematic representation 200 of a method for generating the trained classifier of the telemetry analysis system configured to generate the telemetry indication score. At 210, the system receives input data comprising historical medical information about a plurality of monitored patients. The medical information can include any of the types of data described or otherwise envisioned herein, including but limited to demographics, physiological data, diagnosis information, and telemetry monitoring data (i.e., ECG monitoring data) for the plurality of monitored patients. The input data may be stored in and/or received from one or more databases. The database may be a local and/or remote database. For example, the monitoring center or telemetry analysis system may comprise a database of input data such as a medical health record database among many other types of databases.

The system utilizes the input data to generate a plurality of features related to telemetry monitoring for each of the plurality of previously monitored patients. This can be accomplished by a variety of embodiments for feature identification, extraction, and/or processing. For example, at 220 a data pre-processor or pre-processor module reads input data and generates predictor and outcome variables. The pre-processor may clean the data, evaluate and process waveform quality, extract ECG codes, extract ECG alarms, and perform other analyses.

At 230, a data transformer or transformer module data transformer processes the medical information through the guideline-based decision support tool and to determine whether a condition specified by each decision element has been met. The information generated by the data transformer can include things such as the specific elements present, the percentage of each decision path that has positive induction, the number of paths that have a single indication, the number of trees that are implicated by the data, the maximum level in any decision tree that has a positive indicator, and much more. The outcome of the feature processing is a set of features related to telemetry needs for each of the plurality of previously monitored patients, which thus comprises a training data set that can be utilized to train the classifier.

At step 240, the training data set is utilized to train the classifier. The classifier is trained using the extracted features according to known methods for training a machine learning algorithm. According to an embodiment, a machine learning classifier such as random forest, XGBoost, and/or any other classifier can be used to learn the patient clinical phenotypes and monitoring needs of the patient. Many other classifiers are possible. The classifier can be any machine learning classifier sufficient to utilize the type of input data provided. Thus, following step 240, the system comprises a trained telemetry analysis classifier.

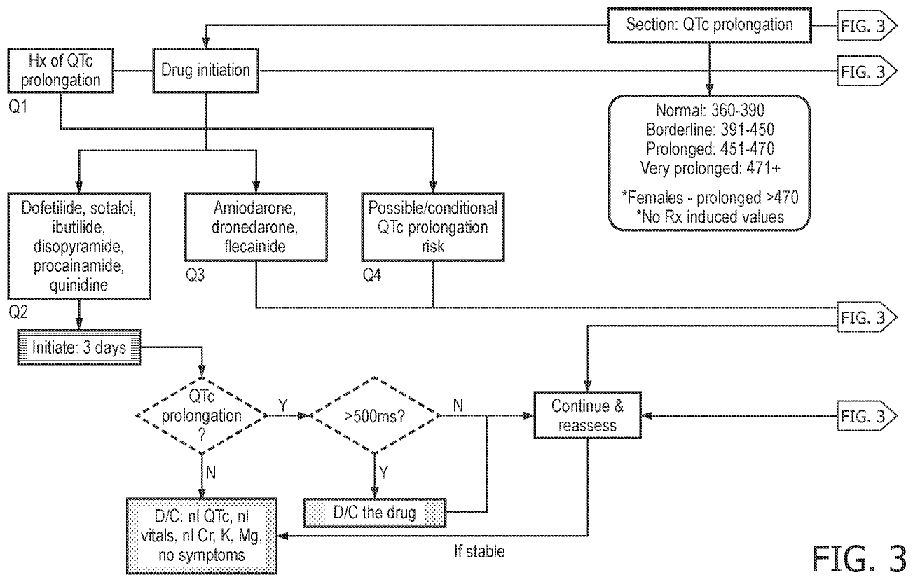

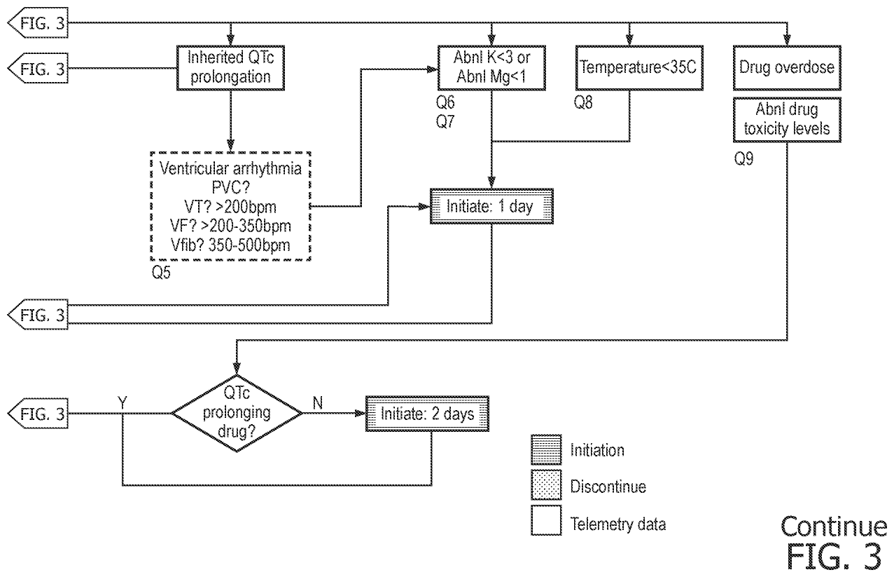

FIGS. 3-10B represent one possible non-limiting example of a decision support tool configuration used by the telemetry analysis system. This embodiment of the decision support tool comprises a plurality of guideline-based decision process trees, in which the guidelines are based at least in part on the AHA guidelines. As described or otherwise envisioned herein, the decision support tool can be configured in many other ways, and if based on issued guidelines those guidelines can be from any source. A decision support tool configuration can, for example, comprise completely customized decision trees or can be updated when new or different guidelines are issued or otherwise available.

In FIGS. 3-10B, each unshaded box in a decision tree describes a criteria or condition that needs to be met in the decision process to reach a decision point. Clinical conditions provide the context needed to infer whether the telemetry use-case fits within a specific decision process. These conditions are predominantly determined from structured data from the EMR or other input data. Decision points are diamonds, which mark decision points that lead to mutually exclusive outcomes, either specific initiation points or discontinuation points. Each figure includes a key to indicate the meaning of a shaded or dotted box. For example, some shapes represent automated evaluation of ECG waveforms and/or past telemetry alarms, other shapes represent missing user input where in some cases the user can be prompted to address whether specific conditions are met, and other shapes represent an ICU/critical care setting. Boxes that state “initiate” indicates sufficient conditions to initiate telemetry and the time until re-assessment is needed. Boxes that state “discontinue” or “D/C” indicates sufficient conditions have been met to discontinue telemetry, unless specific criteria are listed. If specific criteria are listed, monitoring should continue until criteria are met.

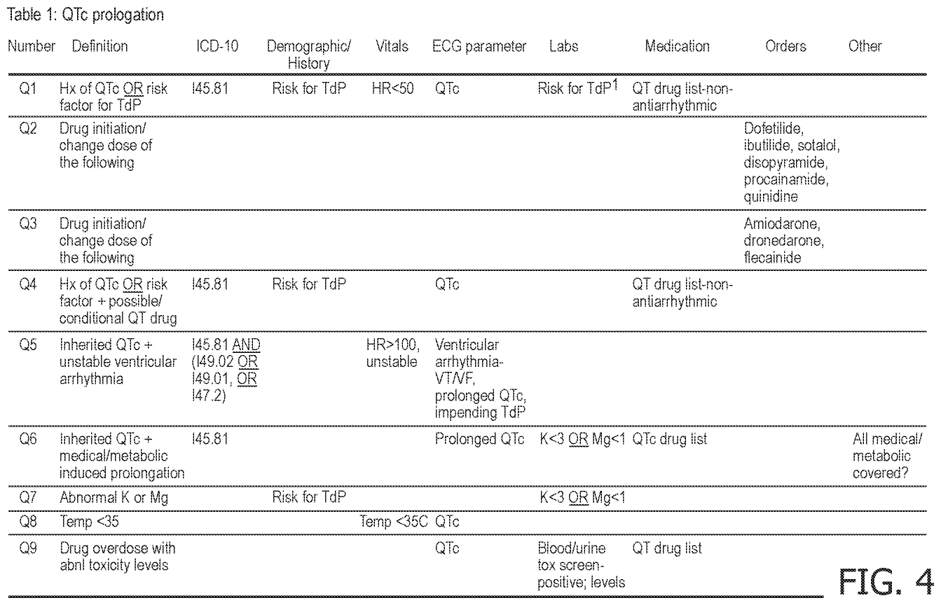

Referring to FIG. 3, in one embodiment, is a QTc prolongation flowchart. The flowchart illustrates all AHA guidelines in medical/surgical units that recommend QTc monitoring. Patients with a history or risk factor for QTc prolongation (e.g., medication or medical history) have specific guidelines for initiation and discontinuation for telemetry. Similarly, lab values and vitals can indicate if telemetry should be initiated. For information regarding data types and details, refer to the numbers in FIG. 4. In this figure, the “Risk for TdP” includes, for example: History—Hx of QTc; Female, advanced age (>65?), MI, HFrEF, LVH, brady-arrhythmia—pause after conversion from AF/flutter to sinus, compensatory pause after PVC, sinus pause, Mobitz II/complete heart block with V rate <40, unexplained QT prolongation in patient/family member, family history of syncope, sudden death, LQTs; Labs—K<3, Mg<1, malnutrition, renal/hepatic failure—BUN/Cr, AST/ALT/ALP, female sex, family history of congenital LQTS, and underlying conditions such as electrolyte abnormalities, renal or hepatic dysfunction, hypothyroidism, heart disease, and bradycardic episodes. “QT drug list” refers to, for example, AZCERT, Inc. which has a list of QT prolonging drugs. “Impending TdP: refers to, for example, sudden bradycardia or long pauses (compensatory pauses after ventricular ectopy, enhanced U waves, T wave alterans, non-sustained polymorphic VT).

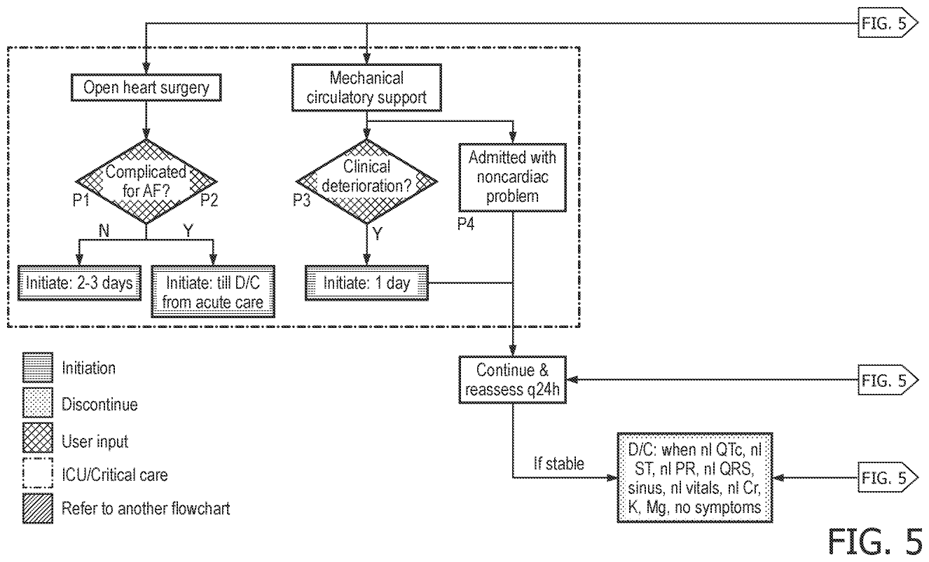

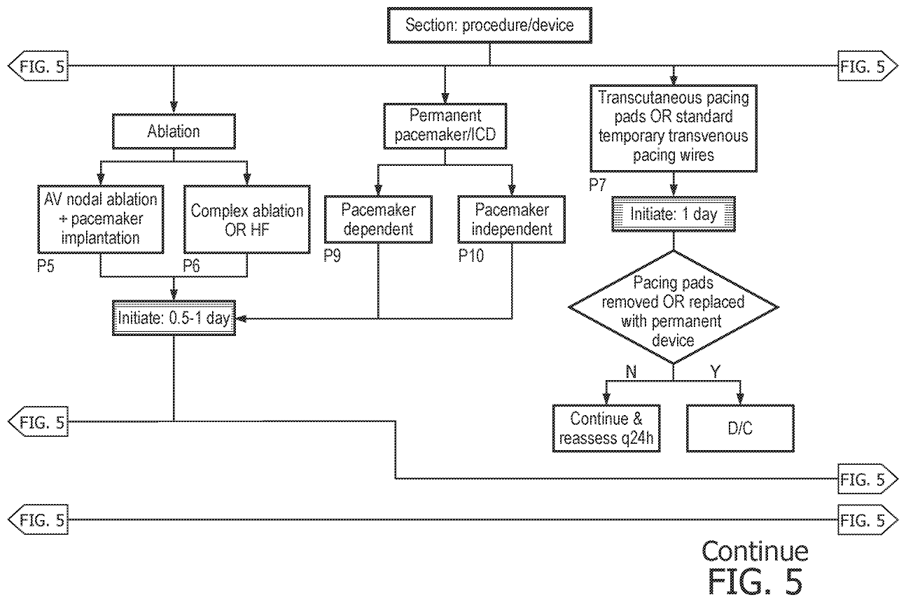

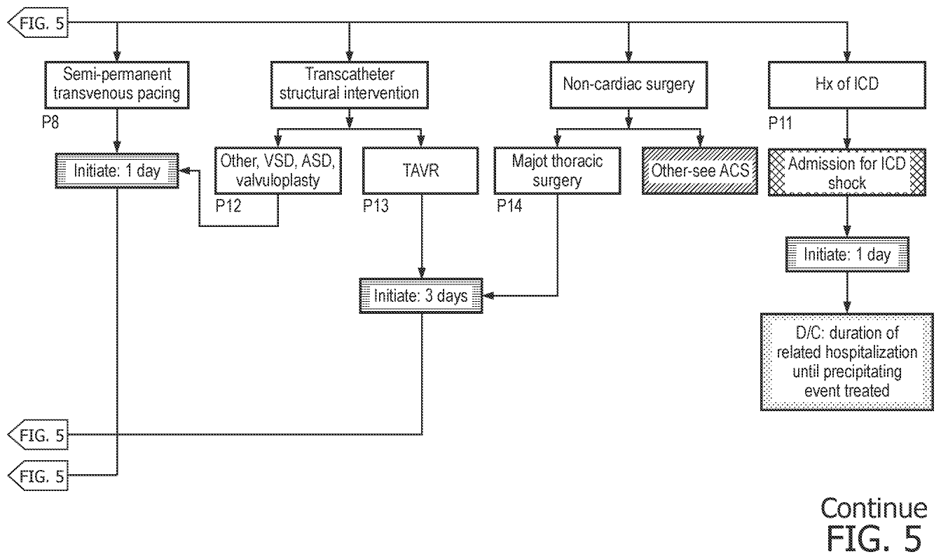

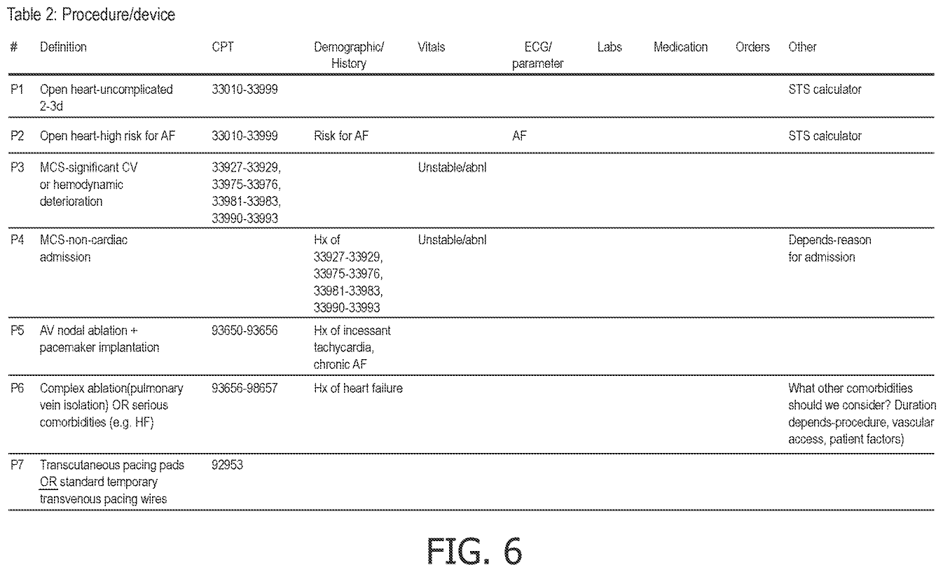

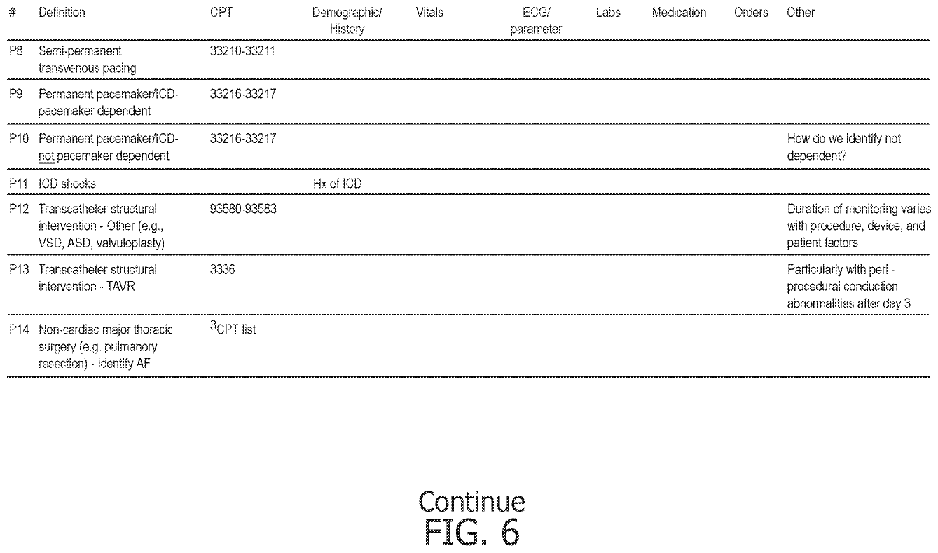

Referring to FIG. 5, in one embodiment, is a procedure or device flowchart. The flowchart illustrates procedures or devices that require telemetry monitoring based on AHA guidelines. Patients who have open-heart surgery, transcatheter structural interventions, ablations, pacing wires, pacemakers, and ICD need telemetry monitoring. The majority of these indications can be identified based on CPT codes. For information regarding data types and details, refer to the numbers in FIG. 6. In this figure, the “STS calculator” refers to, for example, a calculator such as the one available at www.sts.org/resources/risk-calculator. “Risk for AF” refers to, for example: Text (>60 yrs., DM, HTN, CAD, CM, pericardial inflammation, prior MI, CHF, valve/congenital disease, prior cardiac surgery, untreated atrial flutter, thyroid, chronic lung disease, sleep apnea, excessive alcohol/stimulant use, serious illness/infection; Complication for AF—pain, low Hb/Hct, low volume, MI, WBC, low SBP, high LAP, low pH, low HCO3, >65, F, inotrope, low K, low Mg. “CPT” refers to, for example, a need to identify non-cardiac major thoracic surgery list of CPT codes.

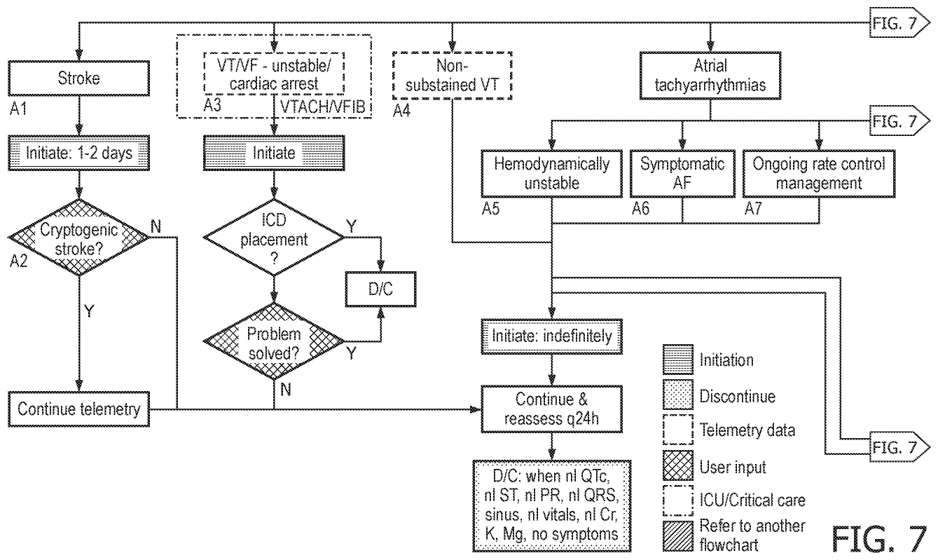

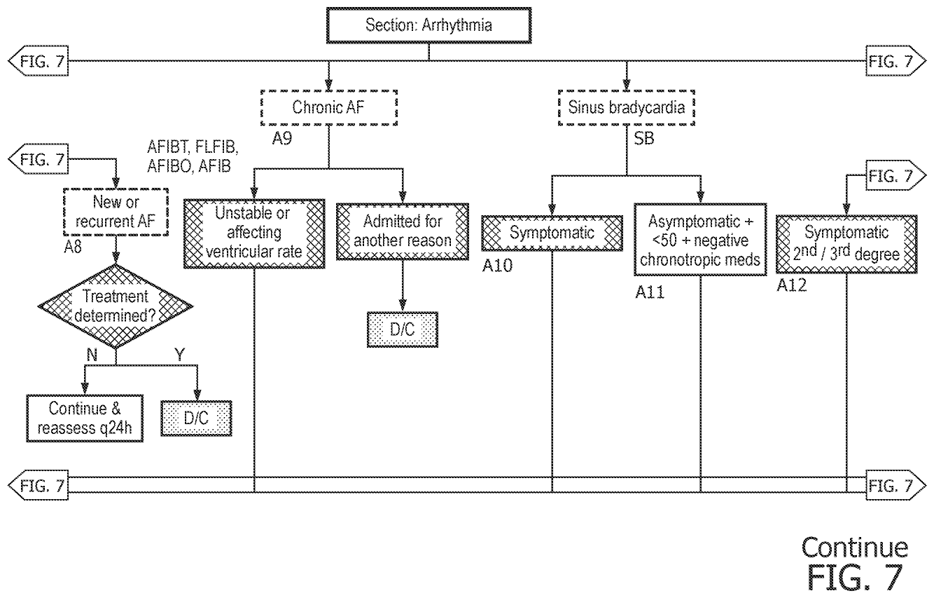

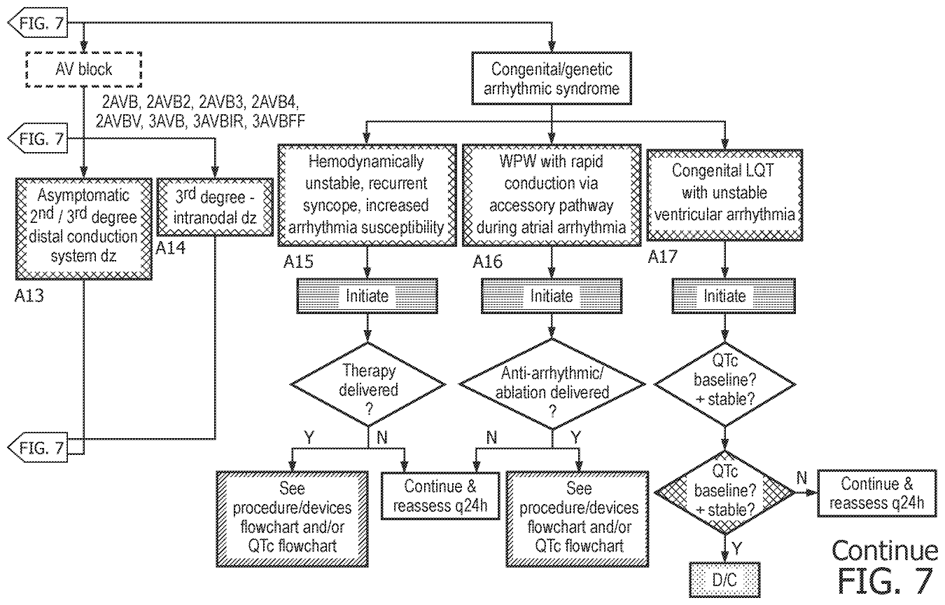

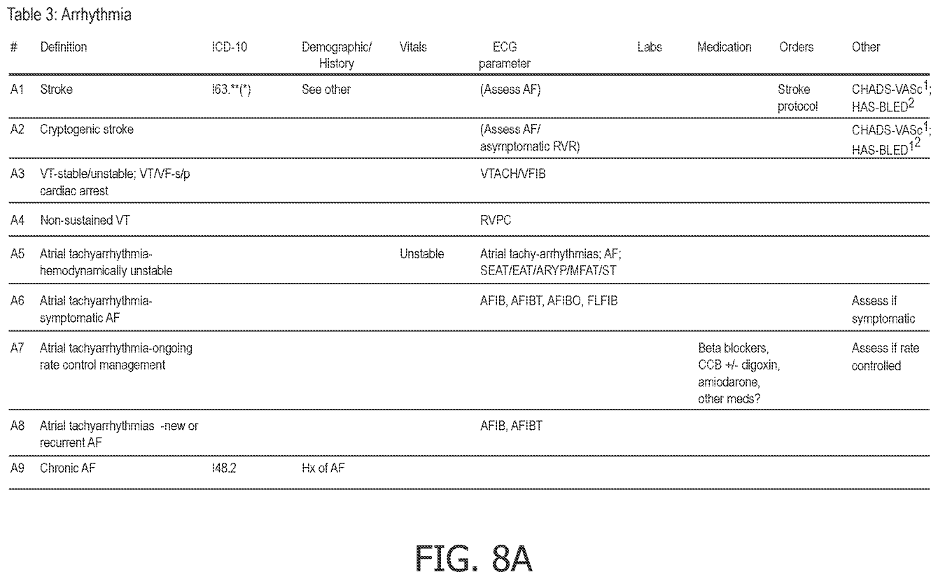

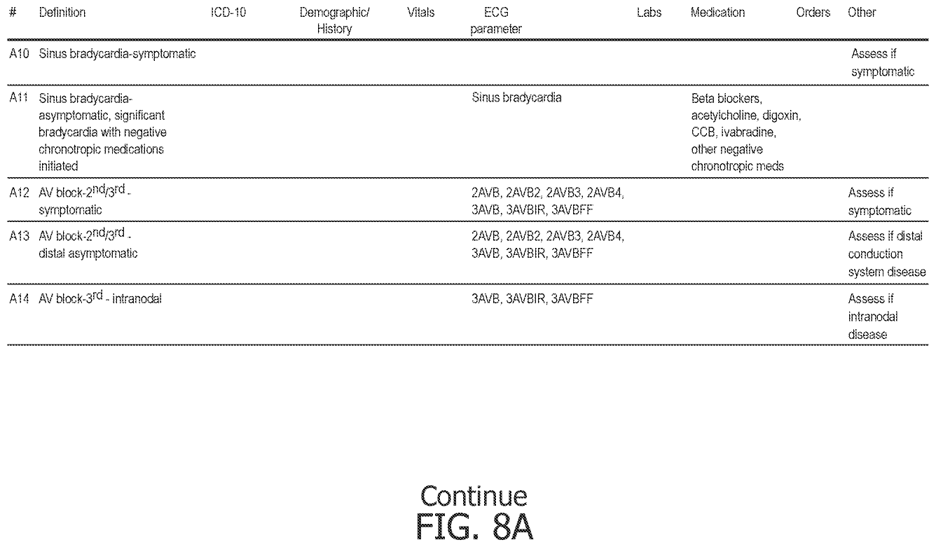

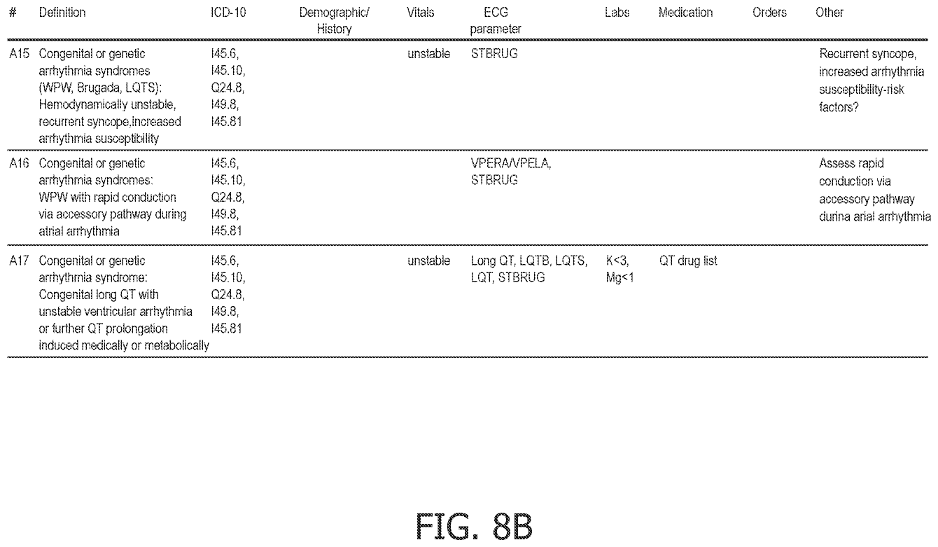

Referring to FIG. 7, in one embodiment, is an arrhythmia flowchart. The flowchart illustrates patients with arrhythmias or risk of arrhythmias that require telemetry monitoring based on AHA guidelines. The majority of these indications can be identified based on algorithms and will require user input. For information regarding data types and details, refer to the numbers in FIGS. 8A and 8B. In FIGS. 8A and 8B, an asterisk can be any integer, and an asterisk in parentheses can be up to 3 digits following “163.” “CHADS-VASc” refers to, for example, a calculation of stroke risk for patients with AF. Variables includes CHF, hypertension, age, stroke/TIA/thromboembolism history, vascular disease history, diabetes history. “HAS-BLED” refers to, for example, an estimate of the risk for major bleeding for patients on anticoagulation to assess risk-benefit in AF care. Variables include hypertension, age >65, stroke history, prior major bleeding/predisposition to bleeding, liver disease, renal disease, labile INR, alcohol use, medication usage predisposing to bleeding.

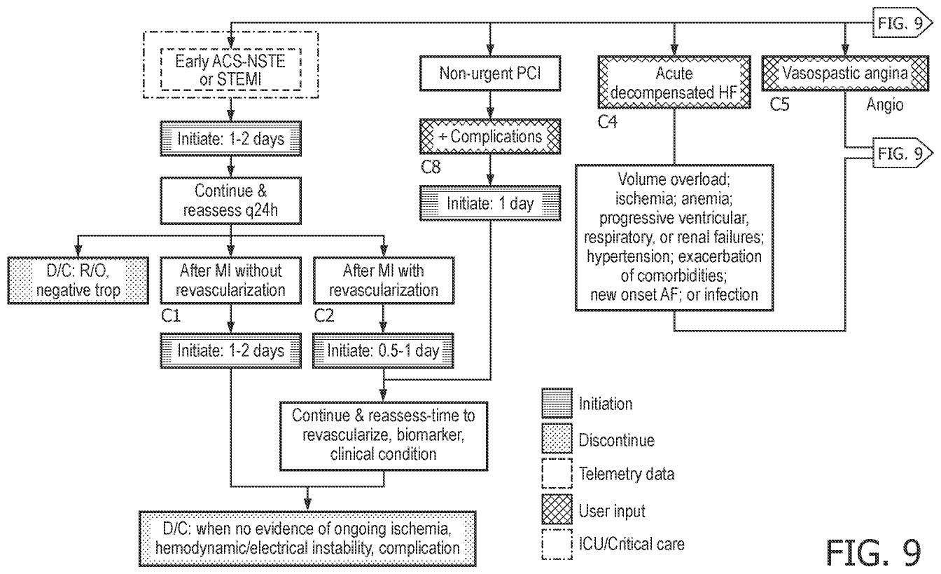

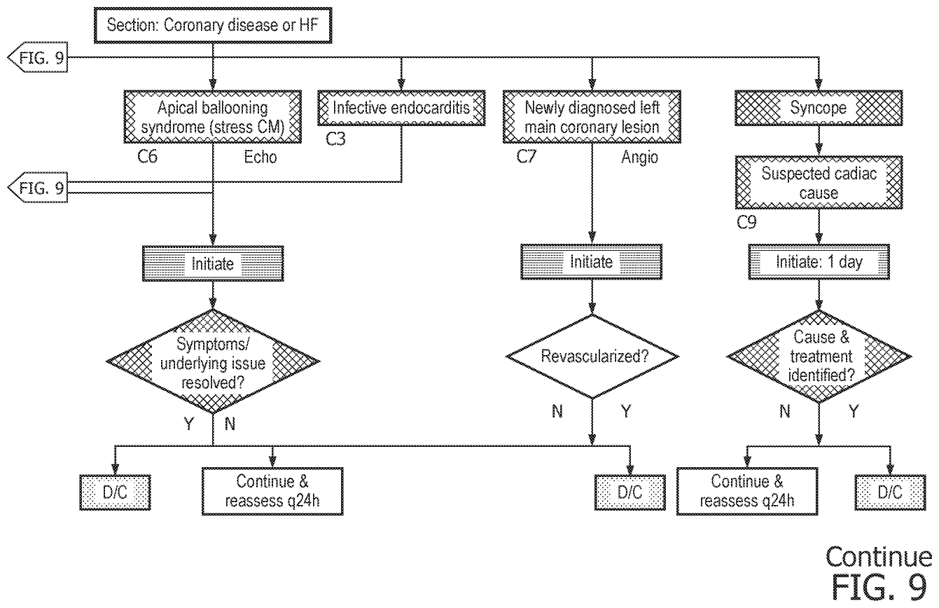

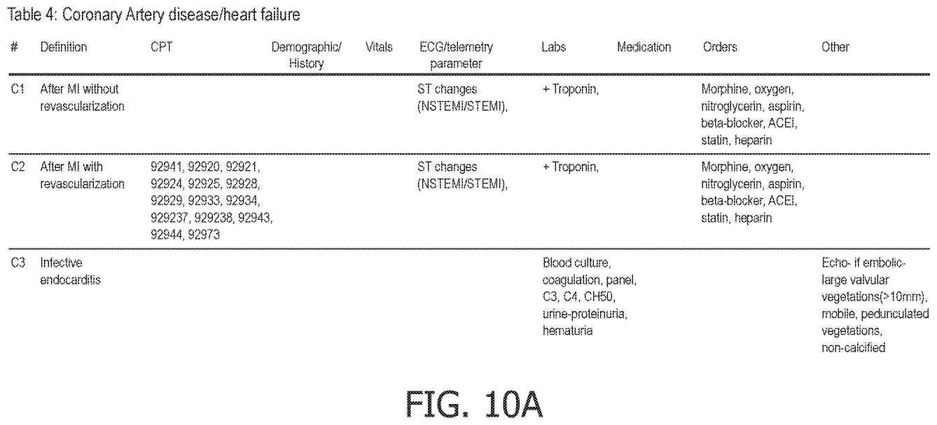

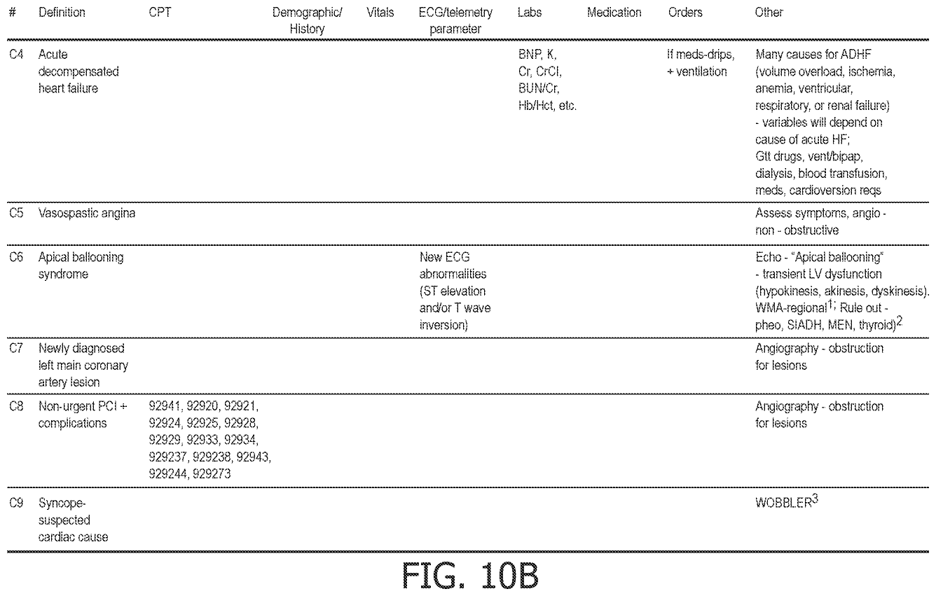

Referring to FIG. 9 is a CAD/HF flowchart. The flowchart illustrates patients with coronary artery disease or heart failure that require telemetry monitoring based on AHA guidelines. Majority of these indications will require user input and other data types. For information regarding data types and details, refer to the numbers in FIGS. 10A and 10B. In FIGS. 10A and 10B, “echo” refers to apical ballooning/Takotsubo syndrome—presence of apical ballooning via echocardiography suggests TCM. “Takotsubo” refers, for example, to a need to rule out pheochromocytoma, myocarditis. Pheochromocytoma—catecholamines (plasma free metanephrines, urinary fractioned metanephrines), CT—absence of pheo. Myocarditis—CBC, ESR, CRP, Rheum screening, CK, Trop, viral Ab titers. “WOBBLER” refers to, for example, Wolff Parkinson White, Obstructive AV pathway, bi-fascicular block, Brugada, Left ventricular hypertrophy, Epsilon wave, Repolarization abnormality.

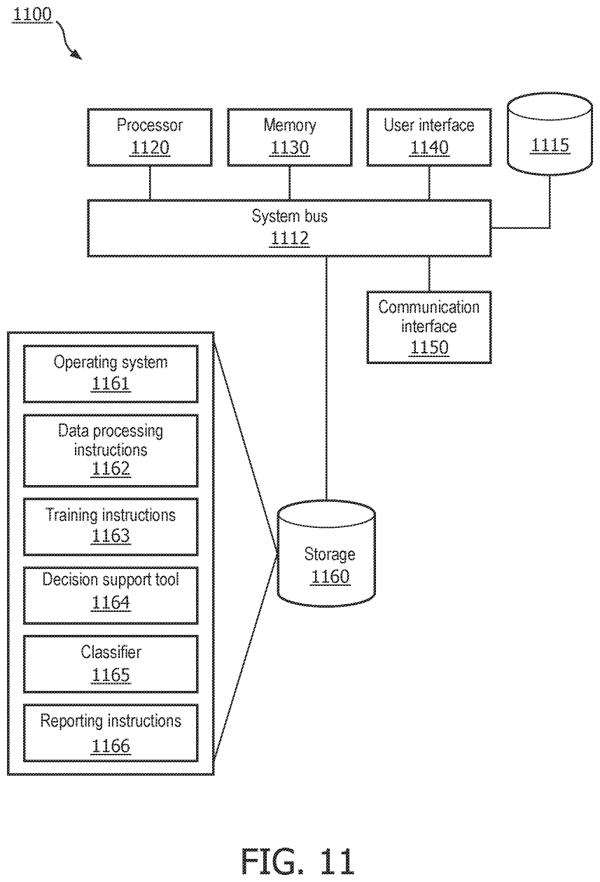

Referring to FIG. 11, in one embodiment, is a schematic representation of a telemetry analysis system 1100. System 1100 may be any of the systems described or otherwise envisioned herein, and may comprise any of the components described or otherwise envisioned herein.

According to an embodiment, system 1100 comprises one or more of a processor 1120, memory 1130, user interface 1140, communications interface 1150, and storage 1160, interconnected via one or more system buses 1112. It will be understood that FIG. 11 constitutes, in some respects, an abstraction and that the actual organization of the components of the system 1100 may be different and more complex than illustrated.

According to an embodiment, system 1100 comprises a processor 1120 capable of executing instructions stored in memory 1130 or storage 1160 or otherwise processing data to, for example, perform one or more steps of the method. Processor 1120 may be formed of one or multiple modules. Processor 1120 may take any suitable form, including but not limited to a microprocessor, microcontroller, multiple microcontrollers, circuitry, field programmable gate array (FPGA), application-specific integrated circuit (ASIC), a single processor, or plural processors.

Memory 1130 can take any suitable form, including a non-volatile memory and/or RAM. The memory 1130 may include various memories such as, for example L1, L2, or L3 cache or system memory. As such, the memory 1130 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 1100. It will be apparent that, in embodiments where the processor implements one or more of the functions described herein in hardware, the software described as corresponding to such functionality in other embodiments may be omitted.

User interface 1140 may include one or more devices for enabling communication with a user. The user interface can be any device or system that allows information to be conveyed and/or received, and may include a display, a mouse, and/or a keyboard for receiving user commands. In some embodiments, user interface 1140 may include a command line interface or graphical user interface that may be presented to a remote terminal via communication interface 1150. The user interface may be located with one or more other components of the system, or may located remote from the system and in communication via a wired and/or wireless communications network.

Communication interface 1150 may include one or more devices for enabling communication with other hardware devices. For example, communication interface 1150 may include a network interface card (NIC) configured to communicate according to the Ethernet protocol. Additionally, communication interface 1150 may implement a TCP/IP stack for communication according to the TCP/IP protocols. Various alternative or additional hardware or configurations for communication interface 1150 will be apparent.

Storage 1160 may include one or more machine-readable storage media such as read-only memory (ROM), random-access memory (RAM), magnetic disk storage media, optical storage media, flash-memory devices, or similar storage media. In various embodiments, storage 1160 may store instructions for execution by processor 1120 or data upon which processor 1120 may operate. For example, storage 1160 may store an operating system 1161 for controlling various operations of system 1100.

It will be apparent that various information described as stored in storage 1160 may be additionally or alternatively stored in memory 1130. In this respect, memory 1130 may also be considered to constitute a storage device and storage 1160 may be considered a memory. Various other arrangements will be apparent. Further, memory 1130 and storage 1160 may both be considered to be non-transitory machine-readable media. As used herein, the term non-transitory will be understood to exclude transitory signals but to include all forms of storage, including both volatile and non-volatile memories.

While system 1100 is shown as including one of each described component, the various components may be duplicated in various embodiments. For example, processor 1120 may include multiple microprocessors that are configured to independently execute the methods described herein or are configured to perform steps or subroutines of the methods described herein such that the multiple processors cooperate to achieve the functionality described herein. Further, where one or more components of system 1100 is implemented in a cloud computing system, the various hardware components may belong to separate physical systems. For example, processor 1120 may include a first processor in a first server and a second processor in a second server. Many other variations and configurations are possible.

According to an embodiment, system 1100 may comprise or be in remote or local communication with a database or data source 1115. Database 1115 may be a single database or data source or multiple. Database 1115 may comprise input data which may be used to train the classifier. The input data may also be the data for a specific patient for which the telemetry analysis is being performed. As an example, the input data can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or current and/or past telemetry data, telemetry alarm related data such as arrhythmia-related alarms (ventricular tachycardia, ventricular bradycardia, atrial fibrillation, asystole, and more), physiologic alarms such as heart rate, respiratory rate, apnea, SpO₂, invasive arterial pressure, noninvasive blood pressure, and more. Many other types of input data are possible. Accordingly, database 1115 may be a EMR database or any other type of database.

According to an embodiment, storage 1160 of system 1100 may store one or more algorithms and/or instructions to carry out one or more functions or steps of the methods described or otherwise envisioned herein. For example, processor 1120 may comprise one or more of data processing instructions 1162, training instructions 1163, decision support tool 1164, classifier 1165, and/or reporting instructions 1116.

According to an embodiment, data processing instructions 1162 direct the system to retrieve and process input data which is used to either: (i) train the classifier 1165 using the training instructions 1163, or (ii) to perform a telemetry analysis for a patient using the decision support tool 1164 and the trained classifier 1165. The data processing instructions 1162 direct the system to, for example, receive or retrieve input data or medical data to be used by the system as needed, such as from database 1115 among many other possible sources. As described above, the input data can comprise a wide variety of input types from a wide variety of sources.

According to an embodiment, the data processing instructions 1162 also direct the system to process the input data to generate a plurality of features related to telemetry monitoring for a cohort of previously monitored patients, which are used to train the classifier. This can be accomplished by a variety of embodiments for feature identification, extraction, and/or processing. For example, a data transformer or transformer module can read input data and generate predictor and outcome variables. A data pre-processor or data processing module can process the predictor and outcome variables using a set of one or more automated rules. The outcome of the feature processing is a set of features related to telemetry monitoring for a cohort of previously monitored patients, which thus comprises a training data set that can be utilized to train the classifier.

According to an embodiment, training instructions 1163 direct the system to utilize the processed data to train the classifier to determine a telemetry indication score for a patient which comprises a probability of whether the patient is likely to meet the telemetry guidelines. The classifier can be any machine learning classifier sufficient to utilize the type of input data provided. Thus, the system comprises a trained classifier 564 configured to determine a telemetry indication score for a patient.

According to an embodiment, decision support tool 1164 comprises telemetry guidelines serving as a mechanism to determine whether a patient should undergo telemetry monitoring. The decision support tool may be of any configuration, design, or format that enables comparison of telemetry input data to elements of the received telemetry guidelines. For example, according to just one embodiment, the decision support tool may be or comprise one or multiple decision trees in which elements of the received telemetry guidelines are formatted into nodes of the decision trees. Thus, medical information received about a patient can be processed through the decision trees determining which elements are met or not met, how well an element is met, and/or other data about the elements in order to create an output of the decision support tool. The guidelines utilized to program or generate the decision support tool can be the American Heart Association guidelines as they are written now or as updated in the future, guidelines from another similar organization, guidelines created by any healthcare professional or healthcare organization, or any combination thereof. Additionally, the guidelines can be personalized or customized for a particular use or location. Other examples of decision support tools are checklists, among many others. Non-limiting examples of a decision support tool are provided elsewhere herein.

According to an embodiment, reporting instructions 1166 direct the system to generate and provide a telemetry indication report for the patient, which includes at least the telemetry indication score generated by the trained classifier of the telemetry analysis system. The telemetry indication report may be a standard format into which output data from the classifier is populated. The telemetry indication report may also be dynamic and formatted or configured based on the output of the classifier. The generated telemetry indication report for the patient is provided via a user interface of the telemetry analysis system. According to an embodiment, the generated telemetry indication report comprises one or more pieces of evidence supporting or informing the telemetry indication score. For example, the evidence may comprise information such as the specific elements of the guidelines present, the percentage of each decision path that has positive induction, the number of paths that have a single indication, the number of decision trees that are implicated by the data, the maximum level in any decision tree that has a positive indicator, and much more. According to an embodiment, the telemetry indication report for the patient further comprises the confidence score. The generated telemetry indication report for the patient can be provided via any mechanism, and via any user interface.

According to an embodiment, the generated telemetry indication report for the patient can then be used by a care provider to determine which patient cases should be reviewed for a telemetry decision, such as whether to initiate telemetry, and whether to continue or discontinue telemetry.

According to an embodiment, the telemetry analysis system is configured to process many thousands or millions of datapoints in the input data used to train the classifier, as well as in the medical information received by the system to perform a telemetry analysis for a patient. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm. Thus, generating a functional and skilled trained classifier comprises a process with a volume of calculation and analysis that a human brain cannot accomplish in a lifetime, or multiple lifetimes.

Similarly, the telemetry analysis system can be configured to continually receive data about a group of patients being monitored, perform the analysis, and provide periodic or continual updates via the telemetry indication report for each patient. This requires the analysis of thousands or millions of datapoints on a continual basis to optimize the telemetry indication reporting, requiring a volume of calculation and analysis that a human brain cannot accomplish in a lifetime.

By providing a quick and thorough telemetry analysis, this novel telemetry analysis system has an enormous positive impact on healthcare compared to prior art systems. As just one example, by providing a more thorough analysis, the system avoids the overuse or misuse of telemetry monitoring, which prevents hospital telemetry services from becoming overburdened or experiencing shortages in telemetry monitoring devices. In addition, the information needed by a human for decision criteria is often incomplete, making interpretation challenging and identification of cases that do not conform to guidelines very difficult. This presents a significant barrier, if not impossibility, for hospitals to rely on understanding, adoption, and continuous execution among care providers as a means of enforcing the valid initiation and continuation of telemetry. Constant re-evaluation is required to determine when a patient can be taken off telemetry, which is very time consuming. Implementing the novel telemetry analysis system significantly improves the sources of data thereby used to make improved analyses regarding telemetry requirements, and avoids utilizing too much time by healthcare professionals, thereby significantly improving care. Additionally, by reducing unnecessary telemetry monitoring, the novel telemetry analysis system can reduce the significant problem of alarm fatigue.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially ofδ shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for generating a telemetry indication score for a patient using a telemetry analysis system, comprising:
receiving, by the telemetry analysis system, medical information about the patient comprising one or more patient demographics, one or more physiological measurements, and/or a patient diagnosis;
analyzing the received medical information using a decision support tool, wherein the decision support tool utilizes telemetry guidelines;
determining, by a trained machine learning algorithm using the results of the decision support tool, a telemetry indication score for the patient comprising a probability of whether the patient is likely to meet the telemetry guidelines; and
providing, via a user interface, a telemetry indication report for the patient, wherein the telemetry indication report comprises the telemetry indication score and further wherein the telemetry indication report comprises evidence supporting the telemetry indication score.

2. The method of claim 1, further comprising the step of programming the decision support tool, comprising: (i) receiving one or more telemetry guidelines; (ii) generating one or more decision trees utilizing the received one or more telemetry guidelines; (iii) optionally adding or modifying, by a healthcare professional, one or more elements of one or more decision trees.

3. The method of claim 1, further comprising the step of training the machine learning algorithm, comprising: (i) receiving a dataset of historical patient data, the dataset comprising for each of a plurality of patient medical information about the patient and telemetry monitoring data for the patient; (ii) extracting, using the decision support tool, a plurality of features from the patient medical information and telemetry monitoring data; and (iii) training (118) the machine learning algorithm using the extracted features.

4. The method of claim 1, wherein the decision support tool comprises a plurality of decision trees each comprising a plurality of decision points derived from telemetry guidelines.

5. The method of claim 1, wherein the decision support tool is configured to determine for the patient, based on the decision tree, one or more elements within the decision tree indicating telemetry and one or more elements within the decision tree contraindicating telemetry.

6. The method of claim 1, wherein the decision support tool is configured to determine for the patient a percentage of elements within the decision tree indicating telemetry.

7. The method of claim 1, wherein the decision support tool is configured to determine which of the plurality of decision trees to utilize for the analysis.

8. The method of claim 1, wherein the one or more physiological measurements comprises one or more vital signs, one or more clinical test results, and/or one or more cognitive assessments.

9. The method of claim 1, wherein the patient diagnosis comprises one or more medical diagnoses, one or more comorbidities, and/or one or more historical medical records.

10. The method of claim 1, wherein the trained machine learning algorithm is further configured to determine a confidence score for the telemetry indication score, and wherein the telemetry indication report for the patient further comprises the confidence score.

11. The method of claim 1, wherein the telemetry indication score is a number between 1 and 100.

12. A telemetry analysis system configured to generate a telemetry indication score for a patient, comprising:
a database comprising medical information about the patient, comprising one or more patient demographics, one or more physiological measurements, and/or a patient diagnosis;
a decision support tool, wherein the decision support tool utilizes telemetry guidelines;
a classifier trained to generate a telemetry indication score for the patient comprising a probability of whether the patient is likely to meet the telemetry guidelines;
a processor configured to: (i) receive the medical information from the database; (ii) direct the decision support tool to analyze the received medical information; (iii) direct the classifier to determine the telemetry indication score for the patient; and (iv) generate a telemetry indication report for the patient; and
a user interface configured to provide the generated telemetry indication report for the patient, wherein the telemetry indication report comprises the telemetry indication score and further wherein the telemetry indication report comprises evidence supporting the telemetry indication score.

13. The system of claim 12, wherein the decision support tool comprises a plurality of decision trees each comprising a plurality of decision points derived from telemetry guidelines.

14. The system of claim 12, wherein the decision support tool is configured to determine for the patient a percentage of elements within the decision tree indicating telemetry.

15. The system of claim 12, wherein the classifier is further configured to determine a confidence score for the telemetry indication score, and wherein the telemetry indication report for the patient further comprises the confidence score.
